(19) United States
(12) Patent Application Publication
(10) Pub. No.: US 2016/0240098 A1
(43) Pub. Date: Aug. 18, 2016

(54) SMART TABLET-BASED NEUROFEEDBACK DEVICE COMBINED WITH COGNITIVE TRAINING, METHOD AND COMPUTER-READABLE MEDIUM THEREOF

(71) Applicant: SEOUL NATIONAL UNIVERSITY R&DB FOUNDATION, Seoul (KR)

(72) Inventors: Min-Sup SHIN, Seoul (KR); Seojin OH, Seoul (KR); Hyejin JEON, Seoul (KR)

(73) Assignee: SEOUL NATIONAL UNIVERSITY R&DB FOUNDATION, Seoul (KR)

(21) Appl. No.: 15/042,579

(22) Filed: Feb. 12, 2016

Related U.S. Application Data
(60) Provisional application No. 62/115,300, filed on Feb. 12, 2015.

(30) Foreign Application Priority Data
Jan. 7, 2016 (KR) 10-2016-0002264

(51) Int. Cl. G09B 19/00; A61B 5/00; G09B 5/00; A61B 5/0482

(52) U.S. Cl. CPC G09B 19/00; A61B 5/0482; A61B 5/742; G09B 5/00

(57) ABSTRACT

A smart tablet-based neurofeedback device combined with cognitive training, a method and a computer readable medium thereof are provided. The neurofeedback combined with cognitive training method includes: receiving a measured beta wave; executing an application for attention training set to a first level; performing a predetermined operation associated with an accomplishment condition of the first level when a value of the received beta wave is larger than a predetermined reference value; and executing a second level including an added accomplishment condition for attention training when the accomplishment condition of the first level is satisfied.

### CROSS-REFERENCE TO RELATED APPLICATIONS

This application claims priority from Korean Patent Application No. 10-2016-0002264, filed on Jan. 7, 2016, in the Korean Intellectual Property Office, and U.S. Provisional Patent Application No. 62/115,300 filed on Feb. 12, 2015 in the USPTO, the disclosures of which are incorporated herein by reference in its entirety.

### BACKGROUND OF THE INVENTION

1. Field of the Invention

Apparatuses and methods consistent with the present disclosure relate to a smart tablet-based neurofeedback device combined with cognitive training, a method and a computer-readable medium thereof, and more particularly, to a smart tablet-based neurofeedback device combined with cognitive training, a method and a computer-readable medium thereof which can perform cognitive training by using a terminal device.

2. Description of the Related Art

With development of electronic technology, various devices have been developed. The developed devices may include even devices that measure brainwaves and use the brainwaves. However, conventional neurofeedback and most programs using the conventional neurofeedback stayed in a passive scheme providing current feedback for changing of brainwaves of a user. Therefore, in regard to the brainwaves, a specific cognitive ability related with a task performance of the user, that is, a training effect to improve attention or memory in practical life was insufficient. In some cases, training devices for improving the cognitive ability of the user are provided, but there was no user customized training system that simultaneously reflects current brainwave control and cognitive performance of the user.

Accordingly, a cognitive training device and a method thereof which can maximize the cognitive training are required.

### SUMMARY OF THE INVENTION

Exemplary embodiments of the present disclosure overcome the above disadvantages and other disadvantages not described above. Also, the present disclosure is not required to overcome the disadvantages described above, and an exemplary embodiment of the present disclosure may not overcome any of the problems described above.

The present disclosure provides a smart tablet-based neurofeedback device combined with cognitive training, a method and a computer-readable medium thereof which can maximize an effect of cognitive training by using a neurofeedback scheme.

According to an aspect of the present disclosure, smart tablet-based neurofeedback method combined with cognitive training includes: receiving a measured beta wave; executing an application for attention training set to a first level; performing a predetermined operation associated with an accomplishment condition of the first level when the value of a received beta wave is larger than a predetermined reference value; and executing a second level including an added accomplishment condition for sustained attention training when the accomplishment condition of the first level is satisfied.

In the performing of the predetermined operation, when the value of the received beta wave is equal to or less than the predetermined reference value, a predetermined penalty may be applied.

The predetermined reference value may be at least one of an average value of the beta wave of a user, which is measured for a predetermined time before executing the application and a reference value adjusted based on a result of neurofeedback based cognitive training which is previously performed.

The neurofeedback combined with cognitive training method may further include: outputting the accomplishment condition as a sound for auditory attention training; displaying a plurality of objects including a target object that satisfies the accomplishment condition; and proceeding to a next step when the target object satisfying the accomplishment condition is selected in response to a selection command of the user.

In the performing of the predetermined operation, when the value of the received beta wave is larger than the predetermined reference value, a hint image of the accomplishment condition may be displayed.

Meanwhile, the neurofeedback combined with cognitive training method may further include displaying an image in which a window including at least one object is cloudily expressed for visual attention and visual memory training, wherein in the performing of the predetermined operation, when the value of the received beta wave is larger than the predetermined reference value, the at least one included object may be shown while the window is wiped.

In addition, the neurofeedback combined with cognitive training method may include: displaying both a target object and an interruption object for visual selective attention training; and counting the number of the target objects selected and the number of the interruption objects selected in response to the selection command of the user, wherein in the performing of the predetermined operation, when the value of the received beta wave is larger than the predetermined reference value, the size of the target object may be adjusted to increase.

Further, the neurofeedback method may further include displaying the received beta wave.

According to another aspect of the present disclosure, a smart tablet-based neurofeedback device combined with cognitive training includes: a communicator receiving a measured beta wave; a processor executing an application for attention training set to a first level; and a display displaying the executed application, wherein the processor may perform a predetermined operation associated with an accomplishment condition of the first level when a value of the received beta wave is larger than a predetermined reference value and execute a second level including an added accomplishment condition for attention training when the accomplishment condition of the first level is satisfied.

When the value of the received beta wave is equal to or less than the predetermined reference value, the processor may apply a predetermined penalty.

The predetermined reference value may be at least one of an average value of the value of the beta wave of a user, which is measured for a predetermined time before executing the application and a reference value adjusted based on a result of neurofeedback based cognitive training which is previously performed.

Meanwhile, the neurofeedback device may further include: a speaker outputting the accomplishment condition as a sound for auditory attention training, wherein the processor may control the display so as to display a plurality of objects including a target object that satisfies the accomplishment condition and proceeds to a next step when the target object satisfying the accomplishment condition is selected in response to a selection command of a user.

When the value of the received beta wave is larger than the predetermined reference value, the processor may display a hint image of the accomplishment condition.

The processor may display an image in which a window including at least one object is cloudily expressed for visual attention and visual memory training, and control the display so as to wipe the at least one included object is shown while the window is wiped when the value of the received beta wave is larger than the predetermined reference value.

The processor may control the display so as to display both a target object and an interruption object for visual selective attention training, count the number of the target objects selected and the number of the interruption objects selected in response to the selection command of the user, and adjust the size of the target object to increase when the value of the received beta wave is larger than the predetermined reference value.

In addition, the processor may control the display so as to display the received beta wave.

Meanwhile, according to yet another aspect of the present disclosure, in a non-transitory readable medium having a program to execute a smart tablet-based neurofeedback device combined with cognitive training, which is recorded therein, the program includes: receiving a measured beta wave; executing an application for attention training set to a first level; performing a predetermined operation associated with an accomplishment condition of the first level when the value of a received beta wave is larger than a predetermined reference value; and executing a second level including an added accomplishment condition for attention training when the accomplishment condition of the first level is satisfied.

According to the diverse exemplary embodiments of the present disclosure, the smart tablet-based neurofeedback device combined with cognitive training, the method and the medium thereof apply the neurofeedback scheme to the cognitive training method to improve user's attention and the ability to control behavior.

Further, the smart tablet-based neurofeedback device combined with cognitive training, the method and the medium thereof are systematic and reflect user's cognitive ability to maintain a training progress with an appropriate intensity and maximize training effects.

In addition, the smart tablet-based neurofeedback device combined with cognitive training, the method and the medium thereof employ a portable device so that everyone can conveniently perform cognitive training in daily life.

Additional and/or other aspects and advantages of the disclosure will be set forth in part in the description which follows and, in part, will be obvious from the description, or may be learned by practice of the disclosure.

### BRIEF DESCRIPTION OF THE DRAWING FIGURES

The above and/or other aspects of the present disclosure will be more apparent by describing certain exemplary embodiments of the present disclosure with reference to the accompanying drawings, in which:

FIG. 1 is a diagram illustrating a neurofeedback combined with cognitive training system according to an exemplary embodiment of the present disclosure;

FIG. 2 is a block diagram of a smart tablet-based neurofeedback device combined with cognitive training according to an exemplary embodiment of the present disclosure;

FIG. 3 is a diagram illustrating a neurofeedback combined with cognitive training process according to a first exemplary embodiment of the present disclosure;

FIG. 4 is a diagram illustrating a neurofeedback combined with cognitive training process according to a second exemplary embodiment of the present disclosure;

FIG. 5 is a diagram illustrating a neurofeedback combined with cognitive training process according to a third exemplary embodiment of the present disclosure;

FIG. 6 is a diagram illustrating a neurofeedback combined with cognitive training process according to a fourth exemplary embodiment of the present disclosure;

FIG. 7 is a diagram illustrating a neurofeedback combined with cognitive training process according to a fifth exemplary embodiment of the present disclosure;

FIG. 8 is a flowchart of a smart tablet-based neurofeedback device combined with cognitive training according to an exemplary embodiment of the present disclosure; and

FIG. 9 is a flowchart illustrating a smart tablet-based neurofeedback device combined with cognitive training according to an exemplary embodiment of the present disclosure.

### DETAILED DESCRIPTION OF THE EXEMPLARY EMBODIMENTS

Hereinafter, exemplary embodiments will be described in more detail with reference to the drawings. The exemplary embodiments disclosed in the specification may be variously modified. A specific exemplary can be illustrated in the drawings and described in detail in the detailed description. However, a specific exemplary embodiment disclosed in the accompanying drawing is used just for easily appreciating various exemplary embodiments of the present disclosure. Therefore, the technical spirit of the present disclosure is limited by the specific exemplary embodiment disclosed in the accompanying drawing and it should be understood that the present disclosure covers all the modifications, equivalents and replacements within the idea and technical scope of the present disclosure. However, in the description of the present disclosure, a detailed explanation and a detailed illustration of known related functions and constitutions may be abbreviated or omitted when it is determined to unnecessarily make the subject matter of the present disclosure obscure.

Terms including an ordinary number, such as first and second, are used for describing various constituent elements, but the constituent elements are not limited by the terms. The terms are used only to discriminate one constituent element from another constituent element.

In the present specification, it should be understood that term “include” indicates that a feature, a number, a step, an operation, a component, a part or the combination thereof described in the specification is present, but does not exclude a possibility of presence or addition of one or more other features, numbers, steps, operations, components, parts or combinations, in advance. It should be understood that, when it is described that an element is “connected to” or “access” another element, the element may be directly connected to or access the another element or a third element may be present therebetween. In contrast, it should be understood that, when it is described that an element “is directly connected to” or “directly accesses” another element, it should be understood that no element is not present between the element and the another element.

Meanwhile, a “module” or “unit” for a component used in the specification performs at least one function or operation. In addition, the “module” or “unit” may perform a function or operation by hardware, software, a combination of the hardware and the software. Further, a plurality of “modules” or a plurality of “units” other than a “module” or “unit” which should be performed in specific hardware or performed in at least one processor may be integrated into at least one module.

Singular expressions used herein include plural expressions unless they have definitely opposite meanings in the context.

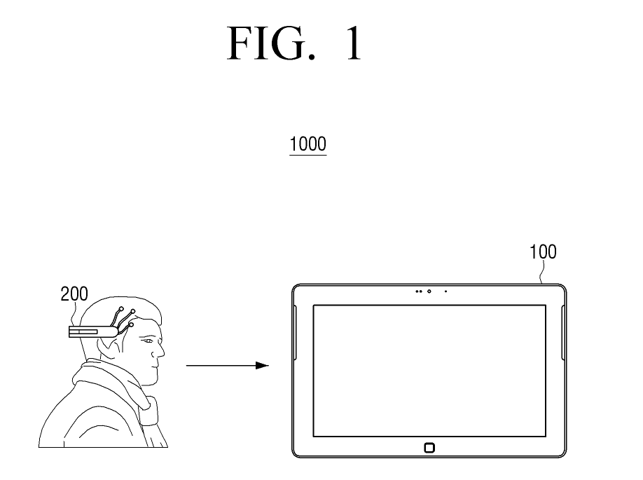

FIG. 1 is a diagram illustrating a neurofeedback combined with cognitive training system according to an exemplary embodiment of the present disclosure.

Referring to FIG. 1, a neurofeedback combined with cognitive training system 1000 is illustrated. The neurofeedback combined with cognitive training system 1000 may include a neurofeedback device combined with cognitive training 100 and a brainwave measurer 200. The brainwave measurer 200 may measure brainwaves and transmit the measured brainwaves to the neurofeedback combined with cognitive training device 100. In human brain, when a signal is transferred between brain nerves in a nervous system, an electric flow called the brainwave or electroencephalography (EEG) is shown. The brainwave may be divided into a delta wave, a theta wave, an alpha wave, a sensory motor rhythm (SMR) wave, a beta wave, or a gamma wave. Among them, the delta wave and the theta wave are associated with sleeping and rest states as a slow-wave activity, the alpha wave is associated with comfortable concentration or the rest state, and an SMR wave which is a frequency between the alpha wave and beta wave is a wave shown when the brain is in a wake-up state, but a mind is stable without tension. In addition, the beta wave as a brainwave output from a conscious activity is associated with maintaining attention and thinking. Meanwhile, the beta wave may be divided into a high beta wave and a low beta wave and the beta wave associated with attention may mean the low beta wave.

The neurofeedback as one type of biofeedback is based on a neurophysiological change to control and normalize an activity pattern of the brain. As an exemplary embodiment, a treatment using the neurofeedback may be used for attention deficit/hyperactivity disorder (ADHD) which is one of most representative pediatric psychiatrist disorders during childhood. In a brainwave study for ADHD children, the theta wave is frequently shown in a frontal lobe area of the brain, while a tendency in which the beta wave is less shown is discovered. In the case of the ADHD children, the beta wave required for awakening or attention is short, while the theta wave activated while relaxation is frequent, and as a result, an activity of a noradrenergic neurotransmitter (noradrenergic activity) decreases, which is required for executing a task and glucose metabolism of the frontal lobe area and a partial subcortical area decreases. Therefore, attention concentration is difficult. Therefore, the neurofeedback training for the ADHD children provides feedback for a neural activity and enforces a change in a required direction to control a specific area of the neural activity by movement.

Some neurofeedback training has been executed up to now, but when the brainwaves of children are primarily changed, passive type training providing only simple visual or auditory feedback therefor is provided. Therefore, the conventional training program shows a problem in that children having an attention problem feel boring and lose motivation easily. Therefore, the present disclosure associates an active cognitive training task to a passive neurofeedback training task to promote the motivation for training the children having the problem in attention, thereby maximizing an effect thereof.

The brainwave measurer 200 may transmit a plurality of measured brainwaves to the neurofeedback combined with cognitive training device 100. Alternatively, the brainwave measurer 200 may transmit the beta wave and the theta wave among the brainwaves to the cognitive training device 100. In some cases, the brainwave measurer 200 may transmit only the beta wave to the neurofeedback combined with cognitive training device 100. In addition, the brainwave measurer 200 may transmit the measured brainwave measured by using a wired/wireless scheme to the neurofeedback combined with cognitive training device 100. As an exemplary embodiment, the brainwave measurer 200 may be manufactured as an earphone type, a headphone type, a wearable device type, or a hood type.

The neurofeedback combined with cognitive training device 100 may receive the brainwaves from the brainwave measurer 200. The neurofeedback training device 100 may detect the beta wave from the received brainwaves. Alternatively, the neurofeedback training device 100 may receive the beta wave from the brainwave measurer 200. The neurofeedback training device 100 receives the beta wave measured for a predetermined time while the user is in a comfortable state (alternatively, a resting state) to detect an average value. The detected average value may be set as a reference value. Alternatively, when a previous training result exists, the previous training result is reflected to set the reference value. For example, when an average value of the beta wave measured in the previous training is 40, the neurofeedback training device 100 may set 40 as the reference value. Alternatively, when the average value of the beta wave measured while the user is in the resting state is 30 and the average value of the beta wave measured in the previous training is 40, the neurofeedback training device 100 may set 35 as the reference value.

The neurofeedback training device 100 may execute an application for the neurofeedback combined with cognitive training. The neurofeedback training device 100 may execute the application and display the received beta wave in one area of the application. The neurofeedback training device 100 may display the value of the received beta wave. In some cases, the neurofeedback training device 100 may display the values of the received beta waves with different colors according to the value of the received beta wave. Meanwhile, the neurofeedback training device 100 may display the value of the beta wave with an absolute value or a relative value. For example, when the value of the beta wave received in the neurofeedback training device 100 is 30, the neurofeedback training device 100 may display the value of the beta wave with the value of 30. That is, the neurofeedback training device 100 may display the beta wave with the absolute value. Alternatively, the average value of the beta wave measured for the predetermined time while the user is in the comfortable state may be 20. The detected average value of the beta wave may become the reference value. Thereafter, when the value of the beta wave received in the neurofeedback training device 100 is 30, the neurofeedback training device 100 may display the value of the beta wave with the value of 10 which is a difference between the reference value and a received value. That is, the neurofeedback training device 100 may display the beta wave with the relative value.

The neurofeedback training device 100 that executes the application for the neurofeedback combined with cognitive training may perform a predetermined operation based on the received beta wave. That is, the neurofeedback training device 100 may perform the predetermined operation when the value of the received beta wave is more than the reference value. For example, the predetermined operation may be an operation of filling an empty pot, an operation of increasing the speed of an object, an operation of providing a hint, an operation of wiping a window, or an operation of increasing the size of the object. Further, when the received beta wave is maintained to be larger than the reference value for a predetermined time, the neurofeedback training device 100 may change a parameter so as to obtain a better result. A detailed operation will be described below.

Since the application for the neurofeedback combined with cognitive training, which is executed by the neurofeedback training device 100 is a game mode application, a concern and a motive of the user may be caused. Further, since the neurofeedback training device 100 uses an average value of a beta wave received for each user or the average value of the beta wave measured in the previous training as the reference value, the neurofeedback training device 100 may perform training optimized for each user. Further, the application may include a plurality of levels. The neurofeedback training device 100 may execute a second level of which a difficulty degree increases when a first level is completed. The second level may be set by increasing a parameter such as a time, the number, or a length for accomplishing a goal.

Therefore, the neurofeedback combined with cognitive training system 1000 may maximize a cognitive training effect by a scheme optimized depending on the user. The neurofeedback training device 100 will be described below.

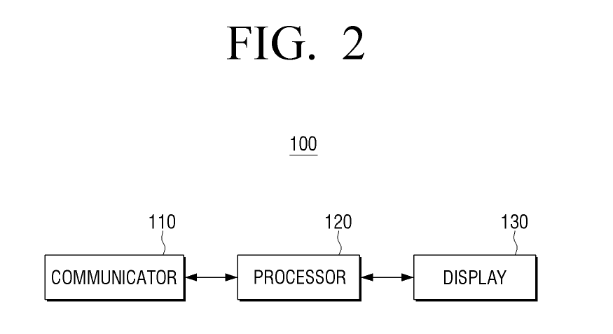

FIG. 2 is a block diagram of a neurofeedback combined with cognitive training device according to an exemplary embodiment of the present disclosure.

Referring to FIG. 2, the neurofeedback training device 100 includes a communicator 110, a processor 120, and a display 130. The attention may include may include processes of arousal, selection, concentration, inhibition (filtering), sustained attention, and attentional shifting. The arousing process may mean a process of switching an unconscious state into a conscious state. The selection process may mean a process of deciding a target for concentration among a plurality of targets. The concentration process may mean a process of being absorbed in the selected target. The inhibition (filtering) process may mean a process of suppressing a concern about an object that interrupts the concentration and concentrating only on the selected target. The sustained attention process may mean a process of maintaining concentration on the selected target for a predetermined time or more. Last, the attentional shifting process may mean shifting the attention on the selected target to another target. In general attention training or cognitive training, the aforementioned process needs to be appropriately trained. Further, visual and auditory training needs to be appropriately provided for efficiency of training.

A training process provided in the neurofeedback combined with cognitive training device may include a process in which only neurofeedback training is performed, a cognitive training process such as a memory, or the like simultaneously with the neurofeedback. In addition, each process may include a plurality of levels.

The brainwave measurer may detect various types of brainwaves by using a preprocessing and frequency analysis algorithm for a measured brainwave signal. In addition, the brainwave measurer may transmit the detected brainwaves to the neurofeedback training device 100. The communicator 110 of the neurofeedback training device 100 receives the measured beta wave. The communicator 110 may receive the plurality of brainwaves including the beta wave from the brainwave measurer 200 and the processor 120 may detect the beta wave. Alternatively, the communicator 110 may receive the measured beta wave and the processor 120 may use the received beta wave. For example, the communicator 110 may include a short-range communication module (not illustrated) for short-range communication. The short-range communication module may perform communication with the brainwave measurer 200, and the like by using a short-range wireless communication network.

The processor 120 may set as the reference value the average value of the value of the beta wave of the user, which is measured by the brainwave measurer 200 for a predetermined time before executing the application. Alternatively, the processor 120 may set as the reference value a value adjusted based on a result of the neurofeedback combined with cognitive training which is previously performed. The processor 120 executes an application for attention training set to the first level. The first level may mean a first level executed during the attention training. That is, the first level may mean an absolute first level, but may mean a relative first level during the training. The processor 120 may perform a predetermined operation associated with an accomplishment condition of the first level when the value of the received beta value exceeds a predetermined reference value. For example, when the accomplishment condition of the first level is a condition to fill three pots, the processor 120 may fill the pot when the value of the received beta wave is larger than a predetermined reference value. As an exemplary embodiment, the processor 120 may fill ⅓ of the pot when the value of the received beta wave is instantaneously larger than the predetermined reference value. Alternatively, the processor 120 may fill ⅓ of the pot when the value of the received beta wave is larger than the predetermined reference value and the increase in value of the beta wave is continued for 0.5 seconds. That is, the neurofeedback training device 100 may set the parameter by various schemes.

The processor 120 may perform the predetermined operation according to the set parameter value when the value of the beta wave is larger than the predetermined reference value. In addition, the processor 120 may execute a second level including the added accomplishment condition when the accomplishment condition of the first level is accomplished. For example, the processor 120 fills the pot when the value of the beta wave is larger than the reference value and when all of three pots are filled, the processor 120 may finish the first level. In addition, the processor 120 may execute the second level. The second level may set a condition to fill 5 pots as the accomplishment condition. That is, when the value of the beta wave is larger than the reference value or the increase in value of the beta wave is continued for the predetermined time, the neurofeedback combined with cognitive training device 100 may give a reward to the user by changing the parameter so as to obtain the better result. Meanwhile, the processor 120 may apply a penalty when the value of the received beta wave is equal to or smaller than the predetermined reference in order to improve the attention of the user. A detailed operation will be described below.

The display 130 may display the executed application and display the value of the received beta wave.

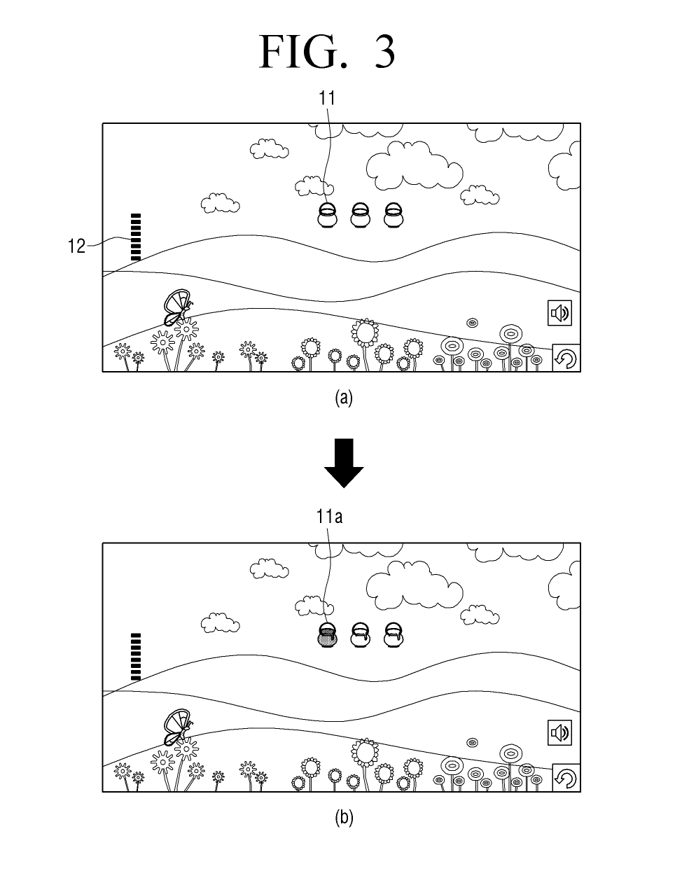

FIG. 3 is a diagram illustrating a neurofeedback combined with cognitive training process according to a first exemplary embodiment of the present disclosure.

Referring to FIG. 3A, an application screen according to the first exemplary embodiment is illustrated. The present disclosure is used not just for enjoying a game but for neurofeedback training. Therefore, the neurofeedback training device may execute a training method which is systematic and depends on the user for cognitive training. The screen illustrated in FIG. 3A shows the process in which only the neurofeedback training is performed. When the neurofeedback training device executes the application, the executed application may be displayed.

The application illustrated in FIG. 3A is an application having filling a honey pot as the accomplishment condition. At a level of the application illustrated in FIG. 3A, a goal is to fill three honey pots. The cognitive training device may display a honey pot 11 to be filled and display the value of the received beta wave at one area of the screen. As described above, the neurofeedback training device may display an absolute value of the received beta wave at one area of the screen and display a relative value by a difference from the reference value at one area of the screen. Alternatively, the neurofeedback training device may display the value of the beta wave with different colors according to the value of the beta wave or duration. The user may concentrate on the value while viewing the screen. The user may concentrate while viewing the honey pot, but may concentrate while viewing another area of the screen. For example, the user may concentrate while viewing a volume button and concentrate while viewing a butterfly. That is, a target object is the honey pot 11, but the cognitive training device according to the first exemplary embodiment may perform a corresponding operation by considering only whether to concentrate without considering a vision of the user.

Referring to FIG. 3B, a screen in which a honey pot 11a is filled is illustrated. The brainwave measurer measures the brainwave of the user to transmit the beta wave or the brainwave including the beta wave to the neurofeedback training device. The neurofeedback training device compares the value of the received beta wave with the predetermined reference value and when the value of the received beta wave is larger than the reference value, the neurofeedback training device may fill the honey pot 11a. As an exemplary embodiment, the neurofeedback training device may fill the honey pot 11a with honey having a predetermined amount when the value of the received beta wave is larger than the reference value. For example, the honey having the predetermined amount may be ⅓ of the honey pot. Alternatively, when the value of the beta wave received for a predetermined time is larger than the reference value, the neurofeedback training device may fill the honey pot 11a with the honey having the predetermined amount. For example, the neurofeedback training device may fill the honey pot 11a with honey of ⅓ when the value of the beta wave is larger than the reference value for 1 second or more. When such an operation is repeated, the neurofeedback training device may fully fill one honey pot 11a. In addition, the neurofeedback training device may fill second and third honey pots with honey. The neurofeedback training device may finish the first level and execute the second level when all of three honey pots are filled. The second level may set filling 6 honey pots as the accomplishment condition. Further, when the beta wave is less than the predetermined reference value, a third level may include a penalty in which the honey of the honey pot decreases. The aforementioned parameter setting is one exemplary embodiment and the parameter may be set by various schemes. The parameter described in an exemplary embodiment given below may also be set by various schemes.

The neurofeedback training device that executes the application illustrated in FIG. 3 may perform visual attention training by using only the neurofeedback. Further, the neurofeedback training device may perform sustained attention training as the number of honey pots to be filled increases according to level up. Since the concentration may be relatively difficult in training using only the neurofeedback, the training using only the neurofeedback may be set with a shorter time than training including interaction.

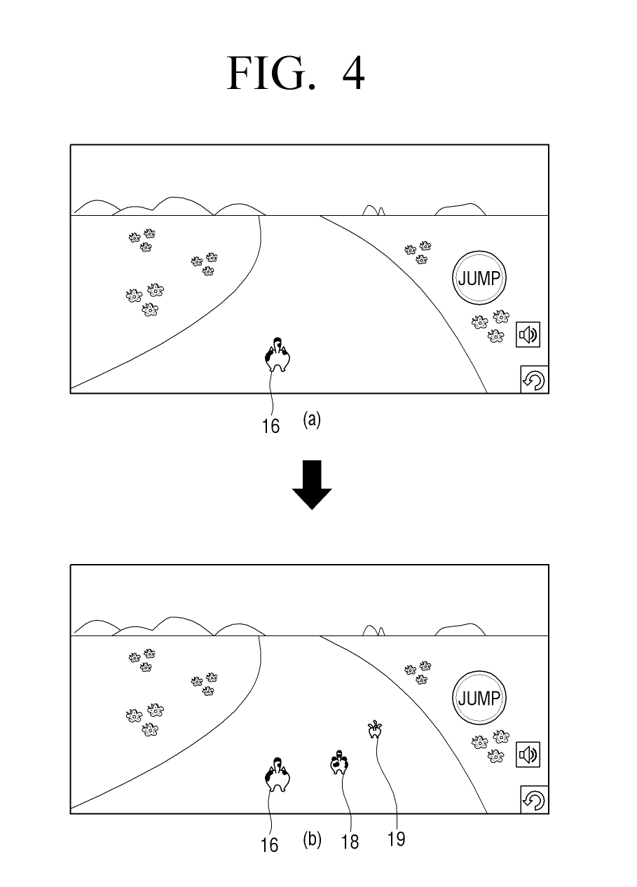

FIG. 4 is a diagram illustrating a neurofeedback training process based on neurofeedback according to a second exemplary embodiment of the present disclosure.

Referring to FIG. 4A, an application screen according to the second exemplary embodiment is illustrated. The screen illustrated in FIG. 4A shows the process in which only the neurofeedback training is performed. The application illustrated in FIG. 4A is an application having allowing a cat to run up to an arrival point as the accomplishment condition. At a level of the application illustrated in FIG. 4A, a goal is to allow the cat to run 1500 m. The neurofeedback training device may display a remaining distance. Further, the neurofeedback training device may display the value of the received beta wave at one area of the screen. The target object is a cat 16 and when the value of the beta wave is larger than the predetermined reference value, the cat may run faster. That is, a predetermined operation associated with the accomplishment condition may be making the cat run faster. The neurofeedback training device may display an obstacle in the meantime. When the cat hits the obstacle, the speed of the cat may decrease. Therefore, the user may jump the cat by touching a jump button. That is, in the case where the obstacle appears, when a jump command is input, the cat jumps to pass the obstacle and the speed of the cat may be maintained.

Referring to FIG. 4B, a target cat 16 that runs a race with other cats 18 and 19 is illustrated. The neurofeedback training device may display other cats 18 and 19 in order to raise an interest of the user. Other cats 18 and 19 may be set to run at a predetermined speed according to the level. That is, when the user may not concentrate and the beta wave is thus smaller than the reference value, the target cat 16 runs more slowly than other cats 18 and 19 to lose the race. When the user concentrates and the beta wave is thus larger than the reference value, the target cat 16 runs faster than other cats 18 and 19 to win the race. The neurofeedback training device may finish the first level and execute the second level when the target cat 16 reaches a destination. The second level may set making the cat run 1900 m as the accomplishment condition. Further, the third level may set making the cat run 2400 m as the accomplishment condition. Meanwhile, as described above, the cognitive training device may include a penalty in which the speed of the target cat 16 decreases when the beta wave is less than a predetermined reference value.

The neurofeedback training device that executes the application illustrated in FIG. 4 may perform the visual attention training by using the neurofeedback. Further, the neurofeedback training device may perform the sustained attention training as a distance up to the destination increases according to level up. In addition, as the obstacle appears, the cat needs to avoid the obstacle through jumping, and as a result, the interaction of the user is required. Therefore, positive neurofeedback training may be performed.

Meanwhile, the neurofeedback training device may further display information including a remaining distance, a training time, a ranking of the target cat 16, the speed of the target cat 16, and the like at one area of the screen.

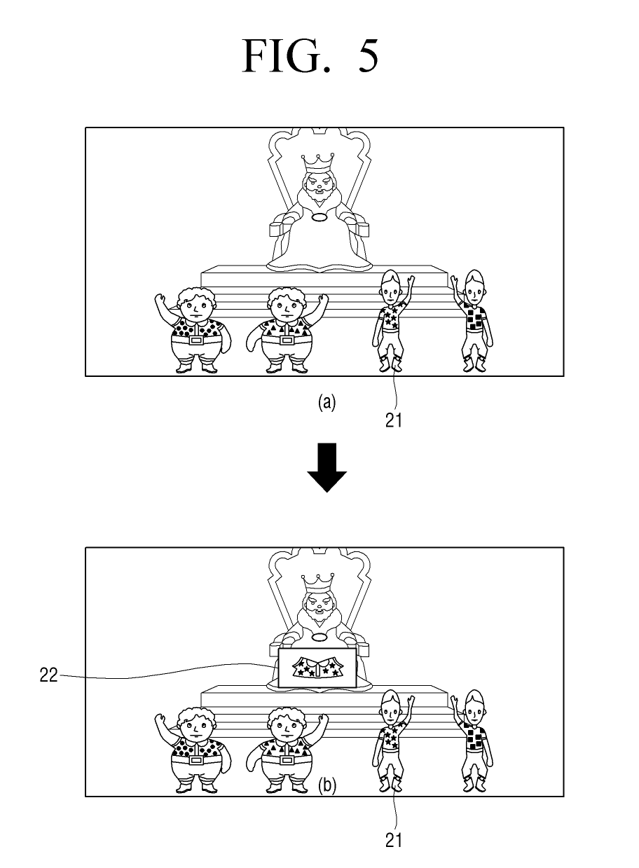

FIG. 5 is a diagram illustrating a neurofeedback combined with cognitive training process according to a third exemplary embodiment of the present disclosure.

Referring to FIG. 5A, an application screen according to the third exemplary embodiment is illustrated. The screen illustrated in FIG. 5A shows the process in which the neurofeedback training and cognitive training including the memory, and the like are simultaneously performed. The application illustrated in FIG. 5A is an application having selecting target object that satisfies a condition in which the target object is output as a sound as the accomplishment condition. The neurofeedback training device includes a speaker to output the accomplishment condition as the sound. For example, the neurofeedback training device may output a command ‘Find a retainer who is slender, wears a starred cloth, and raises a left hand thereof’. The user may select a target object 21 that satisfies the condition after hearing the sound.

The neurofeedback training device may present various conditions according to the level by combining conditions including a body feature, a dress, a worn object, a posture, and the like. For example, the neurofeedback training device may present two conditions such as ‘Find a retainer who is fat and wears a striped cloth’ at the first level. The neurofeedback training device may present three conditions such as ‘Find a retainer who is short, wears a red hat, and has an umbrella’ at the second level. The neurofeedback training device may present four conditions such as ‘Find a retainer who is slender, wears the starred cloth, puts on glasses, and raises the left hand thereof’ at the third level.

Referring to FIG. 5B, a neurofeedback combined with cognitive training device in which a hint 22 is displayed is illustrated. With the concentration of the user, the neurofeedback training device may receive the beta wave which is larger than the reference value. The neurofeedback training device may visually display a hint corresponding to the presented condition when the value of the beta wave is larger than the reference value. For example, when the neurofeedback training device presents a condition ‘Find a retainer who is slender, wears the starred cloth, and raises the left hand thereof’ and the value of the beta wave is larger than the reference value, the neurofeedback training device may display a hint 22 of the starred cloth.

The neurofeedback training device may include more conditions according to the level up. In addition, when the user concentrates well, the neurofeedback training device may display a hint 22 visually representing the presented condition. Meanwhile, the exemplary embodiment illustrated in FIG. 5 may not include a penalty condition.

The neurofeedback training device that executes the application illustrated in FIG. 5 may simultaneously perform the neurofeedback training and the auditory cognitive training including the memory. Further, the neurofeedback training device may perform the sustained attention training as the number of conditions to be memorized increases according to level up. In addition, since the target object 21 needs to be selected, the interaction of the user is required. Therefore, the positive neurofeedback training may be performed.

Meanwhile, the neurofeedback training device may include a plurality of problems at one level and display a presented problem number, a percentage of correct answers, or a percentage of incorrect answers at one area of the screen. For example, in the case where the neurofeedback training device includes 30 problems at the first level, when selection of a first problem is completed, a second problem may be output by moving on a next step.

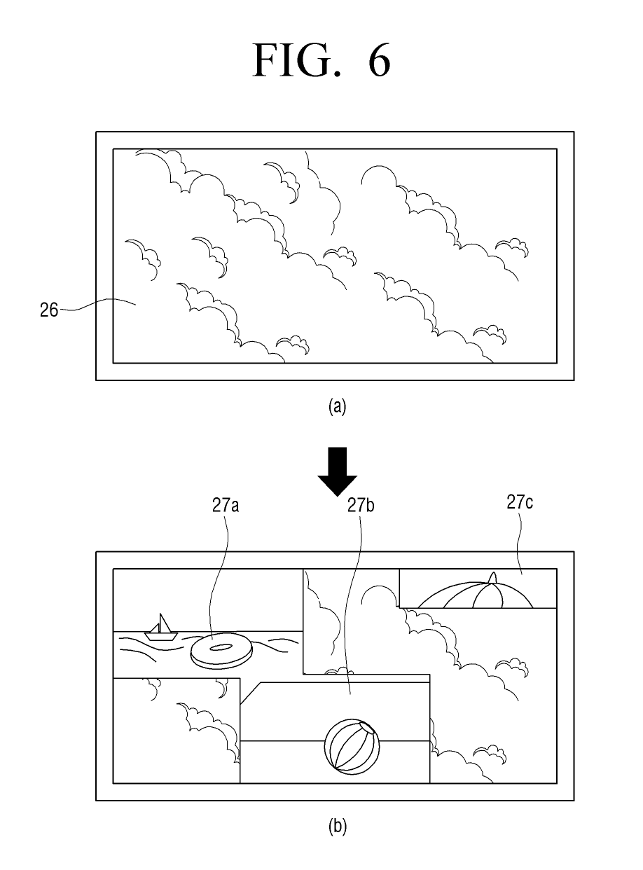

FIG. 6 is a diagram illustrating a neurofeedback combined with cognitive training process according to a fourth exemplary embodiment of the present disclosure.

Referring to FIG. 6A, an application screen according to the fourth exemplary embodiment is illustrated. The screen illustrated in FIG. 6A shows the process in which the neurofeedback training and the visual cognitive training including the memory, and the like are simultaneously performed. An application illustrated in FIG. 6A may include an object. In addition, cloudy dust 26 may be displayed on the included object. When the user concentrates and the cognitive training device receives the beta wave larger than the reference value, the cloudy dust 26 is wiped and the object may be displayed behind the cloudy dust 26. The application illustrated in FIG. 6 is an application having selecting the included object as the accomplishment condition.

Referring to FIG. 6B, a neurofeedback training device in which included objects 27a, 27b, and 27c are displayed is illustrated. With the concentration of the user, the neurofeedback training device may receive the beta wave which is larger than the reference value. The neurofeedback training device may wipe the displayed cloudy dust 26 when the value of the beta wave is larger than the reference value. When the dust 26 is wiped, the object positioned behind the dust 26 may be displayed. The neurofeedback training device may cover the screen with the dust 26 again after a predetermined time. Alternatively, the neurofeedback training device may remove all of the dust 26 and the objects 27a, 27b, and 27c after the predetermined time. In addition, the neurofeedback training device displays multiple objects including the objects 27a, 27b, and 27c positioned behind the dust 26 to allow the user to select the objects 27a, 27b, and 27c positioned behind the dust 26. As an exemplary embodiment, the neurofeedback training device may display a background behind the dust 26 as a beach and display the objects 27a, 27b, and 27c behind the dust as a tube, a beach ball, and a parasol. However, when the background and the objects 27a 27b, and 27c behind the dust are associated with each other, even though the user may not view or memory the objects, the user may easily select the objects, and as a result, the objects may include a polygon or an object not associated with the background.

The neurofeedback training device may include more conditions according to the level up. For example, the first level may include 5 objects, the second level may include 7 objects, and the third level may include an abstract type object which is not associated with the background, that is, the user may not infer in advance based on the background while including 10 objects.

The neurofeedback training device that executes the application illustrated in FIG. 6 may simultaneously perform the neurofeedback training and the visual cognitive training including the memory. Further, the neurofeedback training device may perform the sustained attention training as the number of conditions to be memorized increases according to level up. In addition, since the objects 27a, 27b, and 27c positioned behind the dust 26 need to be selected, the interaction of the user is required. Therefore, the positive neurofeedback training may be performed.

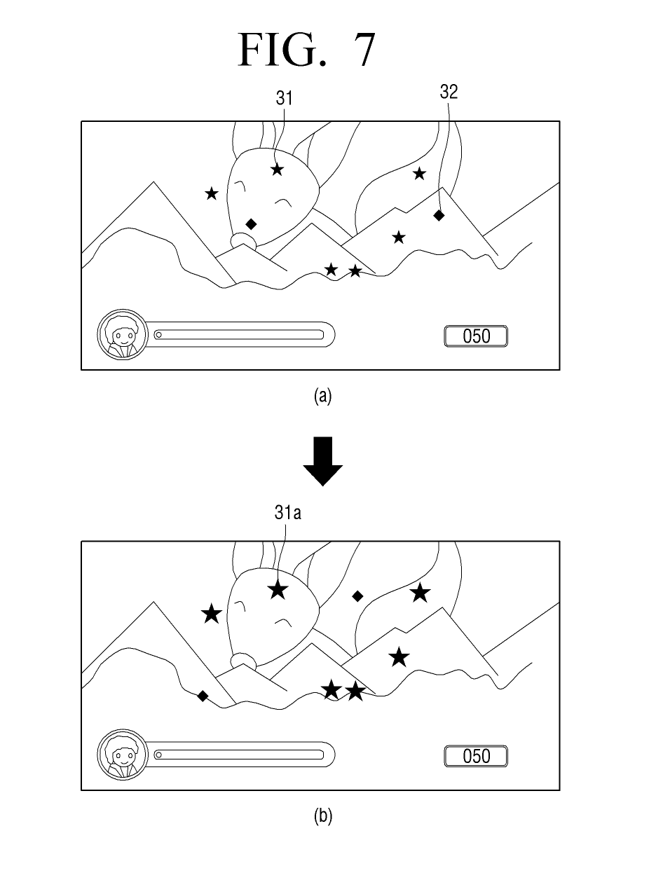

FIG. 7 is a diagram illustrating a neurofeedback combined with cognitive training process according to a fifth exemplary embodiment of the present disclosure.

An application illustrated in FIG. 7A is an application having selecting salts (a starred FIG. 31 as many as a predetermined number as the accomplishment condition. At a level of the application illustrated in FIG. 7A, a goal is to select 50 salts 31. The neurofeedback training device may display the number of remaining salts and display the value of the received beta wave at one area of the screen. The neurofeedback training device that executes the application for the attention training may increase the difficulty degree as the level increases. For example, the neurofeedback training device may set as the goal selecting 50 salts 31 at the first level, 75 salts 31 at the second level, and 100 salts 31 at the third level. In addition, the neurofeedback training device may increase a drop speed of the salt 31 at the second level as compared with the first level and increase the drop speed of the salt 31 at the third level as compared with the second level. Further, the neurofeedback training device may add one interruption object 32 which may be discriminated with a color at the second level and two interruption objects 32 which may be discriminated with the color and a shape at the third level.

Referring to FIG. 7B, a screen in which a predetermined operation is performed according to the beta wave is illustrated. The brainwave measurer measures the brainwave of the user to transmit the beta wave or the brainwave including the beta wave to the neurofeedback training device. The neurofeedback training device compares the value of the receive beta wave with the predetermined reference value and when the value of the received beta wave is larger than the reference value, the neurofeedback training device may increase the size of the salt 31a. That is, when the user concentrates and the value of the beta wave is thus larger than the reference value, the neurofeedback training device may increase the size of to the salt 31a to be selected as a reward.

The neurofeedback training device that executes the application illustrated in FIG. 7 may simultaneously perform the neurofeedback training and the visual cognitive training including selective attention. Further, the neurofeedback training device may perform the sustained attention training as the number of salts to be selected increases according to the level up and as the interruption object appears, the selective attention training and inhibition ability training may be performed and as the drop speed of the salt increases, training that may increase a response speed may be performed.

Up to now, various exemplary embodiments have been described. Here in below, a cognitive training method will be described.

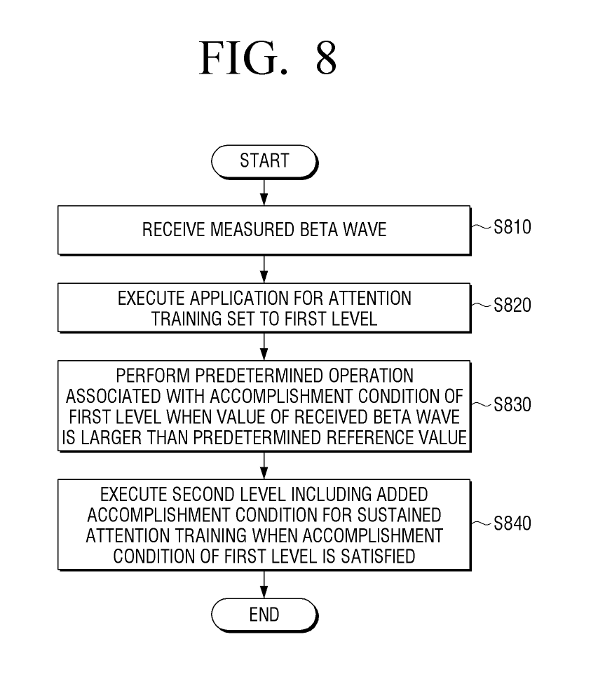

FIG. 8 is a flowchart of a neurofeedback combined with cognitive training method according to an exemplary embodiment of the present disclosure.

Referring to FIG. 8, a neurofeedback training device receives a measured beta wave (S810). A brainwave measurer may measure brainwaves and transmit the measured brainwaves to the neurofeedback training device. The brainwave measurer may transmit the brainwaves including the beta wave and detect the beta wave among the brainwaves received by the neurofeedback training device. Alternatively, the brainwave measurer may detect the beta wave and transmit the detected beta wave to the neurofeedback training device. The neurofeedback training device may execute an application for attention training set to a first level (S820). The application may be used only for neurofeedback training or may simultaneously perform the neurofeedback training and cognitive training.

The neurofeedback training device may perform a predetermined operation associated with an accomplishment condition of the first level when the value of the received beta wave is larger than a predetermined reference value (S830). When the neurofeedback training device performs the predetermined operation, the neurofeedback training device may apply a predetermined penalty when the value of the received beta wave is equal to or smaller than the predetermined reference value. In addition, the predetermined reference value may be at least one of an average value of a beta wave value of a user, which is measured for a predetermined time before executing the application or a reference value adjusted based on a result of neurofeedback combined with cognitive training which is previously performed.

Meanwhile, the neurofeedback combined with cognitive training device may output an accomplishment condition as a sound for auditory attention training and display a hint image of the accomplishment condition when the value of the received beta wave is larger than the predetermined reference value. Alternatively, the neurofeedback combined with cognitive training device may display cloudy dust on a window including an object for visual attention training and visual memory training and display included objects by wiping the cloudy dust when the value of the received beta wave is larger than the predetermined reference value. Alternatively, the neurofeedback combined with cognitive training device may display both a target object and an interruption object for visual selective attention training. Since a detailed process is described above, description of the detailed process will be omitted.

The neurofeedback training device may execute a second level including an added accomplishment condition for the sustained attention when the accomplishment condition of the first level is satisfied. For example, the added accomplishment condition may include an increase in number of honey pots to be filled, an increase in distance up to a destination, an increase in number of condition parameters or an increase in number of problems, an increase in number of target objects, an increase in number of salts to be selected, and the like.

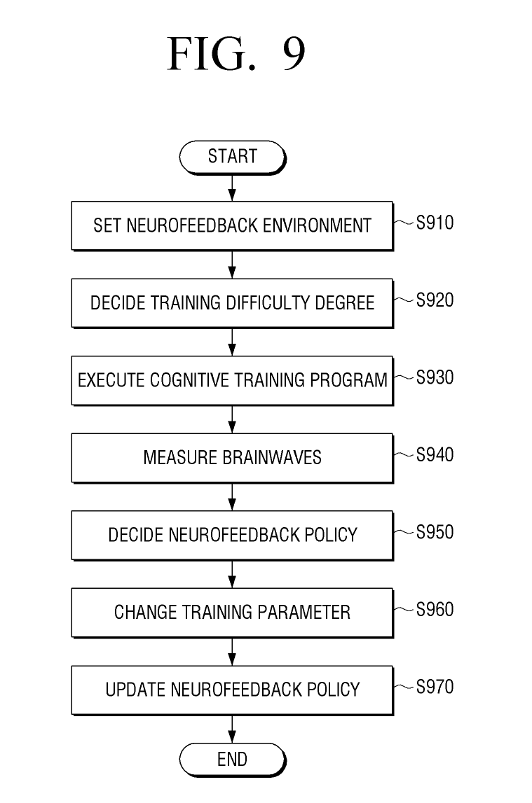

FIG. 9 is a flowchart illustrating a neurofeedback combined with cognitive training process according to an exemplary embodiment of the present disclosure.

A neurofeedback combined with cognitive training device may set a neurofeedback environment (S910). As an exemplary embodiment, a brainwave measurer may measure a brainwave of a user which is in a comfortable state (alternatively, a resting state). In addition, the brainwave measurer may measure a brainwave of a user which is in an exercise training state. The brainwave measurer may transmit the measured brainwave to the neurofeedback training device. The neurofeedback training device compares and analyzes the brainwaves in the comfortable state and the exercise training state to decide a training protocol most suitable for the user. As an exemplary embodiment, the neurofeedback training device may set a training protocol including a beta wave among various brainwaves. Meanwhile, the neurofeedback training device may set a training protocol including a low beta wave among the beta waves. That is, the neurofeedback training device may decide a brainwave among the brainwaves to perform the training and set an environment so as to execute a program to train the decided brainwave. Further, the neurofeedback training device may set a reference value to determine whether the user concentrates.

The neurofeedback combined with cognitive training device may decide a training difficulty degree (S920). In general, the neurofeedback training device may execute an application of a difficulty degree of a first level of an absolute reference. However, the neurofeedback training device may execute an application of a difficulty degree of a second or third level of the absolute reference with respect to a skilled user. As described above, the first level means a level which is relatively first executed and does not particularly mean a level of a first difficulty degree of the absolute reference during a training process that executes the application.

The neurofeedback training device may execute a cognitive training program (S930). The brainwave measurer may measure the brainwaves (S940). For example, the brainwave measurer may be implemented as a head phone type, an earphone type, a wrist band type, or the like. Further, the brainwave measurer may include a microphone for receiving the sound of the user. The brainwave measurer may transmit the measured brainwaves to the neurofeedback training device.

The neurofeedback combined with cognitive training device may decide a neurofeedback policy (S950). The neurofeedback device may compare the value of a brainwave (e.g., a beta wave) of a training goal with a reference value and decide a reward, no feedback, or a penalty (alternatively, punishment). The neurofeedback training device may change a training parameter (S960). The neurofeedback combined with cognitive training device may adjust the training parameter according to the decided neurofeedback policy. For example, the training parameter may include a speed, a value, and the like. The neurofeedback combined with cognitive training device may update the neurofeedback policy (S970). The neurofeedback training device analyzes a result of training which is performed in recent years to adjust the reference value or calculate statistics.

The neurofeedback combined with cognitive training method according to the various exemplary embodiments is implemented as a program to be provided to a processor.

As one example, a non-transitory computer readable medium may be provided, which stores a program that executes a step of receiving a measured beta wave, a step of executing an application for attention training set to a first level, a step of performing a predetermined operation associated with an accomplishment condition of the first level when the value of a received beta wave is larger than a predetermined reference value, and a step of executing a second level including an added accomplishment condition for sustained attention training when the accomplishment condition of the first level is satisfied.

The non-transitory computer readable medium means not media which stores data for a short instant, such as a register, a cache, a memory, and the like but a medium that semipermanently stores data and is readable by an apparatus. In detail, the various applications or programs may be provided while being stored in the non-transitory readable media such as a CD, a DVD, a hard disk, a blu-ray disk, a USB, a memory card, a ROM, and the like.

Preferred exemplary embodiments of the present disclosure have been illustrated and described above, but the present disclosure is not limited to the above-described specific embodiments, it is obvious that various modifications may be made by those skilled in the art, to which the present disclosure pertains without departing from the gist of the present disclosure, which is claimed in the claims, and such modified embodiments should not be individually understood from the technical spirit or prospect of the present disclosure.

What is claimed is:

1. A neurofeedback device combined with cognitive training, the neurofeedback training method comprising:
receiving a measured beta wave;
executing an application for attention training set to a first level;
performing a predetermined operation associated with an accomplishment condition of the first level when a value of the received beta wave is larger than a predetermined reference value; and
executing a second level including an added accomplishment condition for sustained attention training when the accomplishment condition of the first level is satisfied.

2. The neurofeedback combined with cognitive training method as claimed in claim 1, wherein the performing of the predetermined operation comprises to apply a predetermined penalty when the value of the received beta wave is equal to or less than the predetermined reference value.

3. The neurofeedback combined with cognitive training method as claimed in claim 1, wherein the predetermined reference value is at least one of an average value of the beta wave of a user, which is measured for a predetermined time before executing the application and a reference value adjusted based on a result of neurofeedback based cognitive training which is previously performed.

4. The neurofeedback combined with cognitive training method as claimed in claim 1, further comprising:
outputting the accomplishment condition as a sound for auditory attention training;
displaying a plurality of objects including a target object that satisfies the accomplishment condition; and
proceeding to a next step when the target object satisfying the accomplishment condition is selected in response to a selection command of the user.

5. The neurofeedback combined with cognitive training method as claimed in claim 4, wherein the performing of the predetermined operation comprises to display a hint image of the accomplishment condition when the value of the received beta wave is larger than the predetermined reference value.

6. The neurofeedback combined with cognitive training method as claimed in claim 1, further comprising:
displaying an image in which a window including at least one object is cloudily expressed for visual attention and visual memory training,
wherein, when the value of the received beta wave is larger than the predetermined reference value, the performing of the predetermined operation comprises to show the at least one included object while the window is wiped.

7. The neurofeedback combined with cognitive training method as claimed in claim 1, comprising:
displaying both a target object and an interruption object for visual selective attention training; and
counting the number of the target objects selected and the number of the interruption objects selected in response to the selection command of the user,
wherein the performing of the predetermined operation comprises to adjust to increase the size of the target object when the value of the received beta wave is larger than the predetermined reference value.

8. The neurofeedback combined with cognitive training method as claimed in claim 1, further comprising:
displaying the received beta wave.

9. A neurofeedback device combined with cognitive training, the neurofeedback training device comprising:
a communicator receiving a measured beta wave;
a processor executing an application for attention training set to a first level; and
a display displaying the executed application,
wherein the processor is configured to:
perform a predetermined operation associated with an accomplishment condition of the first level when a value of the received beta wave is larger than a predetermined reference value and execute a second level including an added accomplishment condition for sustained attention training when the accomplishment condition of the first level is satisfied.

10. The neurofeedback combined with cognitive training device as claimed in claim 9, wherein when the value of the received beta wave is equal to or less than the predetermined reference value, the processor applies a predetermined penalty.

11. The neurofeedback combined with cognitive training device as claimed in claim 9, wherein the predetermined reference value is at least one of an average value of the beta wave of a user, which is measured for a predetermined time before executing the application and a reference value adjusted based on a result of neurofeedback based cognitive training which is previously performed.

12. The neurofeedback combined with cognitive training device as claimed in claim 9, further comprising:
a speaker outputting the accomplishment condition as a sound for auditory attention training,
wherein the processor controls the display so as to display a plurality of objects including a target object that satisfies the accomplishment condition and proceeds to a next step when the target object satisfying the accomplishment condition is selected in response to a selection command of a user.

13. The neurofeedback combined with cognitive training device as claimed in claim 12, wherein when the value of the received beta wave is larger than the predetermined reference value, the processor displays a hint image of the accomplishment condition.

14. The neurofeedback combined with cognitive training device as claimed in claim 9, wherein the processor displays an image in which a window including at least one object is cloudily expressed for visual attention and visual memory training, and controls the display so as to wipe the at least one included object is shown while the window is wiped when the value of the received beta wave is larger than the predetermined reference value.

15. The neurofeedback combined with cognitive training device as claimed in claim 9, wherein the processor controls the display so as to display both a target object and an interruption object for visual selective attention training, counts the number of the target objects selected and the number of the interruption objects selected in response to the selection command of the user, and adjusts the size of the target object to increase when the value of the received beta wave is larger than the predetermined reference value.

16. The neurofeedback combined with cognitive training device as claimed in claim 9, wherein the processor controls the display so as to display the received beta wave.

17. A non-transitory readable medium having a program to execute a neurofeedback combined with cognitive training method, which is recorded therein, the program comprising:
receiving a measured beta wave;
executing an application for attention training set to a first level;
performing a predetermined operation associated with an accomplishment condition of the first level when the value of the received beta wave is larger than a predetermined reference value; and
executing a second level including an added accomplishment condition for sustained attention training when the accomplishment condition of the first level is satisfied.
